(19) United States
(12) Patent Application Publication
(10) Pub. No.: US 2024/0416125 A1
(43) Pub. Date: Dec. 19, 2024

(54) Method for Non-Invasive or Minimally-Invasive Stimulation of Deep Brain Targets

(71) Applicant: CARNEGIE MELLON UNIVERSITY, Pittsburgh, PA (US)

(72) Inventors: Mats Forssell, Pittsburgh, PA (US); Yuxin Guo, Pittsburgh, PA (US); Yuhyun Lee, Pittsburgh, PA (US); Vishal Jain, Pittsburgh, PA (US); Chaitanya Goswami, Pittsburgh, PA (US); Ariel Feldman, Pittsburgh, PA (US); Neil Ashim Mehta, Pittsburgh, PA (US); Boyle C. Cheng, Pittsburgh, PA (US); Pulkit Grover, Pittsburgh, PA (US)

(73) Assignee: CARNEGIE MELLON UNIVERSITY, Pittsburgh, PA (US)

(21) Appl. No.: 18/742,524

(22) Filed: Jun. 13, 2024

Related U.S. Application Data
(60) Provisional application No. 63/472,729, filed on Jun. 13, 2023.

(51) Int. Cl. A61N 1/05; A61N 1/36; A61N 1/378; A61N 2/00

(52) U.S. Cl. CPC A61N 1/36082; A61N 1/0534; A61N 1/0546; A61N 1/0548; A61N 1/36128

(57) ABSTRACT

Disclosed herein is a method for delivery stimulation to targeted deep brain structures by supplementing existing method using external electrodes with transnasal electrodes, disposed, for example, in the olfactory cleft and within the hyenoid sinus, or, more broadly, in the nasal cavity and/or in the sinuses, including, but not limited to, the frontal, ethmoidal, sphenoid sinus, etc. The method allows stimulation and/or sensing in deep brain regions which can be inaccessible from the scalp or for which existing methods are ineffective in targeting. The method can also be used for power delivery to implants that might be placed inside the brain or on its surface.

### RELATED APPLICATIONS

This application claims the benefit of U.S. Provisional Patent Application No. 63/472,729, filed Jun. 13, 2023, the contents of which are hereby incorporated herein in their entirety.

### GOVERNMENT INTEREST

This invention was made with U.S. government support under contract N65236-19-C-8017 awarded by the United States Department of the Navy. The U.S. government has certain rights in this invention.

### BACKGROUND

Non-invasive/minimally-invasive deep-brain stimulation is of great interest, for example, the brain's “reward circuit” consists of multiple cortical and subcortical (deep-brain) nodes, as shown in FIG. 1 which include the orbitofrontal cortex (OFC), hippocampus, amygdala, nucleus accumbens, thalamus, etc. This circuitry processes reward and punishment, and is implicated in several conditions, for example, depression, suicidal ideation, PTSD, and substance use disorder.

Recently, for patients suffering from conditions that are treatment-resistant, a promising treatment alternative is emerging: implanted electrodes in nodes of the reward circuit to electrically stimulate these brain areas and successfully treat them. For example, deep-brain stimulation (DBS) has been used to stimulate Brodmann Area 25 (subgenual cingulate area) for patients with treatment-resistant depression. Such treatments are typically effective over a long term (i.e., 3-6 years) and do not induce significant adverse effects. In another example, the lateral OFC was stimulated and acute improvement in mood-state was observed in patients with moderate-to-severe depression. DBS treatments are also used experimentally for patients with brain injuries and those in vegetative/minimally-conscious state, with good success rates, and some of these targets are outside of the standard reward circuit, but still lie in the deep brain.

However, DBS requires a sophisticated surgical procedure to implant the system (including electrodes, battery, and the associated circuitry) which carries risk of intracranial hemorrhage and infection. Moreover, the stimulation target region cannot be changed once the electrodes are implanted inside the brain. Non-invasive techniques such as transcranial magnetic stimulation (TMS), transcranial focused ultrasound stimulation (tFUS), and transcranial electrical stimulation (TES) have lower risk and are steerable, but are also limited in their depth, resolution, and effect. TMS and TES effects are relatively shallow (i.e., 2 to 4 cm below the cortical surface), and are unable to target deep brain structures, especially near the ventral side of the brain. The mechanisms of tFUS are not well understood, and its effects are thought to cause potentiation or modulation of firing rate rather than direct stimulation. TES passes electrical currents to directly stimulate neurons resulting, for example, in motor evoked potentials when targeting the motor cortex. TES has been used for decades, thus its safety profile and mechanisms are comparatively better understood. However, these non-invasive techniques traditionally inject energy from the top of the head (i.e., through the scalp), and therefore require higher amplitudes to affect neurons at large depths. This leads to stimulation of undesired shallow targets (which lowers focality), which can cause unintended side effects and intolerable scalp pain or even tissue damage.

### SUMMARY OF THE INVENTION

The embodiments described herein provide the capability to improve the targeted stimulation of deep brain tissues. Disclosed herein is a method for placing electrodes transnasally, as shown in FIG. 2, in the olfactory cleft and within the sphenoid sinus, or, more broadly, transnasally or transorally, in the nasal cavity, the nasopharynx, and/or in the sinuses, including, but not limited to, the frontal, ethmoidal, sphenoid sinus, etc., to sense and/or stimulate in deep brain regions which can be inaccessible from the scalp or for which existing methods are ineffective in targeting such deep brain regions.

In some embodiments, the techniques can harness low-resistivity pathways to inject/receive more energy. In some embodiments, pathways can include foramina (holes). In some embodiments, foramina can include, but are not limited to, foramina in the cribriform plate and/or in the skull base and the foramen lacerum. In some embodiments, pathways can include the thinner skull. In some embodiments, the thinner skull can include, but are not limited to, at some of the skull-base locations. In some embodiments, pathways from a thinner skull can include, but are not limited to, from the sphenoid sinus to the pituitary or the brain.

The described neuromodulation embodiments may include techniques for electrical stimulation, where electrodes placed at various locations can inject optimized current amplitudes for creation of focal, high-intensity current fields for neurostimulation. In some embodiments, electrodes can deliver high frequency currents for treating tumors. In some embodiments, electrodes can be used for sensing, where they can be used to make inferences about the deep brain activity including, but not limited to detection of dysfunctional neural structures or changes in the signaling pathways. In other embodiments, the system can be used to deliver power to deep-brain implants, or deliver light (e.g., for photoswitching drugs, near-infrared light for sensing (e.g., using fNIRS), ultrasound delivery for stimulation, etc.)

The transnasally/orally-placed electrodes may be used in a stand-alone fashion or may supplement electrodes placed on the scalp.

The method may be used to supplement and further optimize current injection patterns for efficient and focal targeting of reward circuit nodes, such as the method described in U.S. patent application Ser. No. 18/021,257, entitled “Method for Focused Transcranial Current Stimulation”, which discloses a method for optimizing the placement of electrodes on the scalp for therapeutic purposes.

In placing electrodes inside the olfactory cleft (i.e., under the cribriform plate) and within the sphenoid sinus, the physical proximity to deep brain structures and the high-conductive pathways offered by thin bones and foramina in the cribriform plate may be exploited to target stimulation of deep brain structures.

The disclosed method creates larger and more focal fields at deep brain targets than traditional scalp electrode configurations. Moreover, the stimulation is steerable, i.e., the clinicians can choose and change the target after electrode placement, which is an advantage over implantable deep brain stimulation. Lastly, the transnasal/oral placement of the electrodes may be used to enhance existing sensing modalities.

When using scalp electrodes and transnasal electrodes together, the distance (from the electrodes) at which neural responses can be evoked is increased compared to cases using only transnasal/oral electrodes or only electrodes on the scalp.

The presence and the spatial pattern of the cribriform foramina (the holes in the cribriform plate) can be harnessed such that the electrode placement can be optimized to generate maximum current density in preferred directions at chosen targets. By using the electrodes under the cribriform plate exclusively, one can target nearby regions in the brain as well as nerves. Specifically, using spatial arrangement of currents and use of current waveforms that work with neural dynamics, following structures can be targeted: the terminal nerve (also called “Cranial Nerve 0”) without affecting the olfactory bulb (by generating local patterns), the olfactory bulb and/or the terminal nerve but not lateral OFC/PFC (the parts of the brain closest to the ethmoid bone) and the olfactory bulb, the terminal nerve, and/or regions within the lateral OFC. Differences in dynamics of regions as well as spatial and spatiotemporal interference could be used to stimulate regions farther from the electrodes without stimulating regions close by.

By using electrodes under the cribriform plate in conjunction with those placed in the frontal sinus, the ethmoid sinus, etc. and the scalp, sub-cm subregions of the lateral and medial OFC and Brodmann area 25, among other regions, can be targeted.

### BRIEF DESCRIPTION OF THE DRAWINGS

FIG. 1 is an illustration showing important nodes in the reward circuitry of the brain.

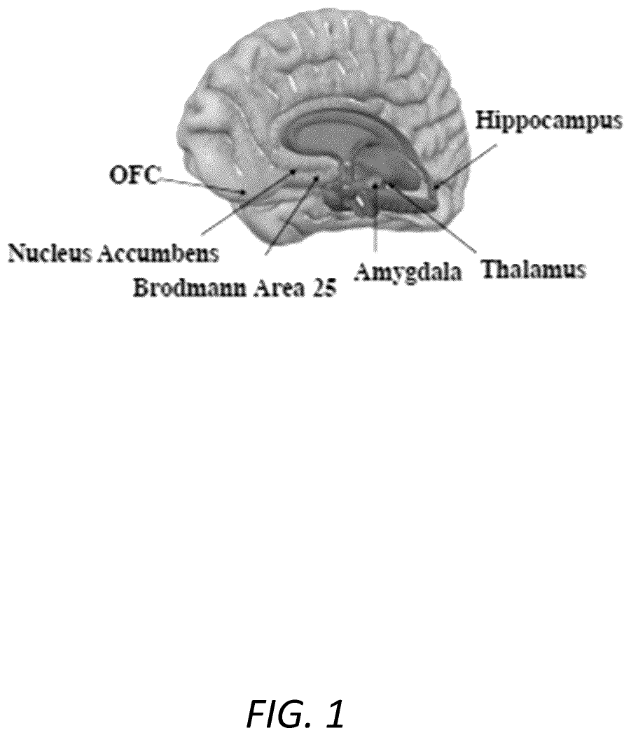

FIG. 2 shows placement of the electrodes in the nasal areas.

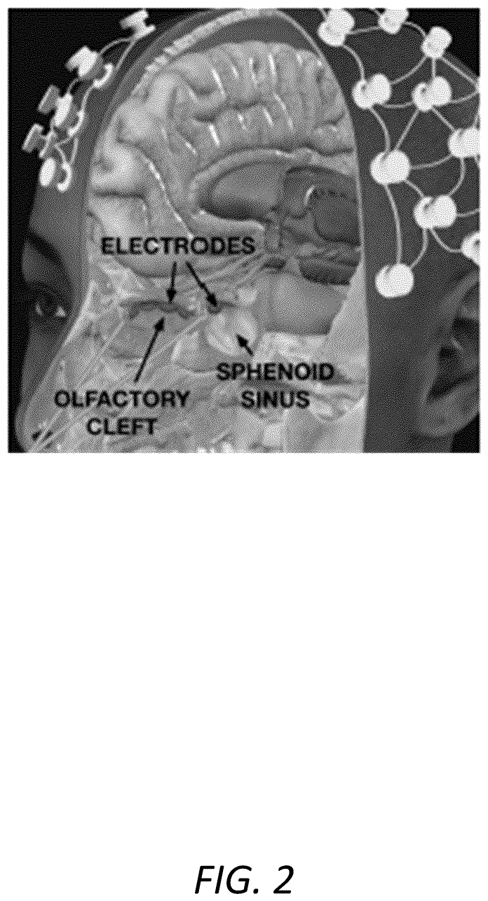

FIG. 3 is an illustration of the segmentation of head and brain tissue after the addition of ethmoid foramina.

FIG. 4 is an illustration showing the placement of electrodes in the olfactory cleft.

FIG. 5 is an illustration showing the placement of electrodes in the sphenoid sinus.

FIG. 6 is an illustration of the magnitude of the electric field created by scalp+transnasal and scalp-only patterns.

FIG. 7 is a graph showing the percentage of voxels with electric field magnitude greater than that of the ROI at an OFC target.

FIG. 8 is a graph showing the percentage of total field contained in increasing volumes around ROI at an OFC target.

FIG. 9 is a graph showing the percentage of voxels around ROI that contain 50% of the total field magnitude at an OFC target.

FIG. 10 illustrates electric field magnitude targeting BA25 using scalp+transnasal configurations.

FIG. 11 illustrates electric field magnitude targeting BA25 using scalp only configurations.

FIG. 12 is a graph showing the percentage of voxels with electric field magnitude greater than that of an ROI at a BA25 target.

FIG. 13 is a graph showing the percentage of total field contained in increasing volumes around the ROI at a BA25 target.

FIG. 14 is a graph showing the Percentage of voxels around the ROI that contain 50% of the total field magnitude at a BA25 target.

FIG. 15 illustrates the coefficient of variation across the 10 different head models for every voxel inside the brain computed using a Monte Carlo simulation analysis.

FIG. 16 is a graph showing the electric field in the direction of the sEEG probe generated by a scalp+transnasal pattern and a scalp-only pattern measured in cadaver brain.

FIG. 17 is a graph showing the recorded electric field in a cadaver brain versus distance from the deepest recording site.

### DETAILED DESCRIPTION

The disclosed methods include a set of novel techniques to perform deep brain sensing and/or stimulation through an intact skull around the brain. In some embodiments, techniques can utilize electrodes/sensing and/or stimulation units placed on the scalp, and/or those placed transnasally in the olfactory cleft (under the ethmoid bone's cribriform plate) and/or, more broadly, in the nasal cavity; and/or in the sinuses, including, but not limited to, the frontal, ethmoidal, sphenoid sinus, etc., to sense and/or stimulate in deep brain regions which can be inaccessible from the scalp.

In some embodiments, the techniques can harness low-resistivity pathways to inject/receive more energy. In some embodiments, pathways can include foramina (holes). In some embodiments, foramina can include, but are not limited to, foramina in the cribriform plate and/or in the skull base. In some embodiments, pathways can include thinner skull. In some embodiments, thinner skull can include, but are not limited to, at some of the skull-base locations. In some embodiments, pathways from a thinner skull can include, but are not limited to, from the sphenoid sinus to the pituitary, the hypothalamus, or other regions of the deep brain.

The olfactory cleft is close to the ventral side of the brain and can be accessed through the nose with endoscopic guidance. This space is directly below the cribriform plate, which is a thin and complicated bone structure with foramina (i.e., holes) that allow blood vessels and olfactory nerves to pass through. Directly above the cribriform plate is the olfactory bulb, which is situated below the medial orbitofrontal cortex of the brain.

The sphenoid sinus is an air-filled structure proximal to the pituitary gland and the cranial nerves. The sphenoid sinus is accessible with minimally-invasive procedures. ENT surgeons routinely access the olfactory space, for example, for sinus surgeries. Local anesthetics such as tetracaine are frequently used to reduce discomfort. Endoscopic-guided transnasal/oral positioning is relatively easy and quick (requiring approximately 15 minutes for experienced ENT surgeons, after preparing the olfactory mucosa/tissue) to perform. Placement in the sphenoid sinus is, however, harder. The access to the sinus is through the sphenoid ostium, an opening into the sinus that is accessible transnasally, but could require soft-tissue manipulation to insert an electrode.

In some embodiments, electrodes can be made to make good contact with sinus walls in air-filled sinuses to inject large currents (and/or sense). Good contact between the electrodes and the sinus walls can be accomplished by gently pushing the electrode (e.g., in a non-limiting example, transnasally in the sphenoid sinus). In one embodiment the electrodes may be deployed on a balloon, (e.g., a balloon similar to that used in balloon angioplasty) that can be inflated once inside the sinus to make good contact with electrode walls. In some embodiments, the electrodes may be configured as a 2- or 3-dimensional array of electrodes. In some embodiments, electrodes deployed in the sinuses can be battery driven or could be powered wirelessly.

Ethmoid sinuses have small air cells inside ethmoidal labyrinths, which may make good contact between electrodes and sinus walls difficult. In some embodiments, good contact between the electrodes with the sinus walls can be accomplished by careful bone removal inside these sinuses.

Implants in sphenoidal sinus could easily access the pituitary through electric currents (separated by 3-5 mm). Similarly, using electric currents, implants in the frontal and ethmoidal sinuses can access frontal and orbitofrontal brain regions. The combination of electrodes in ethmoidal sinus and scalp can potentially stimulate the amygdala and the anterior hippocampus (2-3 cm), nuclei in the hypothalamus, mammillary bodies, nucleus accumbens, etc.

In some embodiments, due to the steerability of electric currents, with transnasal/oral, scalp, and/or sinus electrodes, one can choose and change the target after placement of electrodes. This is in contrast to existing implantable deep-brain stimulators, which have minimal flexibility in target location post-implantation. In some embodiments, steerability can provide clinicians with flexibility to adjust treatments after electrode placement, something that is infeasible with invasive deep-brain stimulation.

In some embodiments, beamforming optimization algorithms, including, but not limited to, advances in the algorithm disclosed in U.S. patent application Ser. No. 18/021,257, can be used to determine the placement and activation pattern of transnasal/sinus and/or scalp electrodes. In some embodiments, data-driven machine learning models can be trained for optimal electrode placement prediction. Variations of anatomical details (including, but not limited to, position and/or size of cribriform foramina) across the population can be encoded in the training process as a set of variables. With a robust model trained with a comprehensive dataset, optimal electrode placement may be predicted for any new individual with unseen anatomy.

In some embodiments, search algorithms can be used to further fine-tune the spatial and temporal pattern of injected current to induce desired response in targeted regions.

Methods described herein can be used for non-invasive sensing and/or stimulation, including, but not limited to, using electronic, magnetic, light, thermal, and/or ultrasound waves. In some embodiments, a combination of these techniques can be used if any single technique is providing insufficient currents, and/or due to reaching pain thresholds inside the head and/or on the scalp. These waves can also be used for delivering power to implants in the brain or on its surface.

Simulations were conducted as a proof-of-concept with various configurations of transnasal electrodes. Empirically-derived confirmation of electrical stimulation was confirmed in a number of different human cadaveric studies. The invention is thus explained in the context of these simulations.

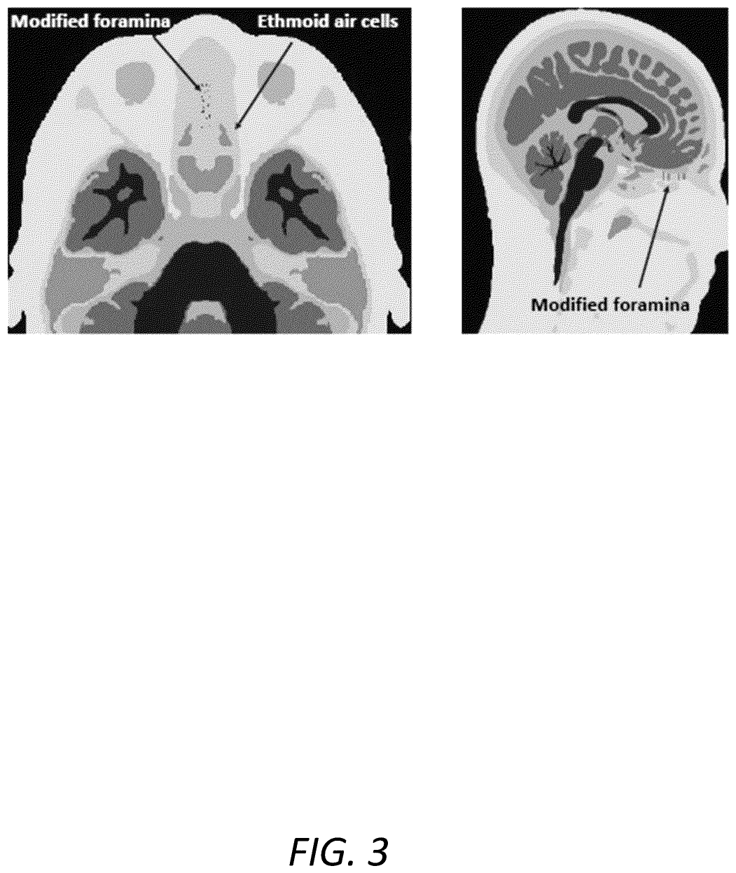

Simulations were conducted on a head model with realistic geometry and tissue conductivity. The New York head model, with 0.5 mm resolution was used to construct the forward matrix, which relates the applied current at electrodes to the electric field in brain voxels. Since the transnasal electrodes are placed inside the olfactory cleft, realistic modeling of the ethmoid bone anatomy was necessary. However, anatomical details are typically not preserved after tissue auto-segmentation, so the New York head tissue masks were manually modified, specifically the cribriform plate, with reference to existing literature that informs the area and number of the cribriform plate foramina. The size of the foramina was assumed to be 0.25, 0.5, and 1 mm², with 8 on the left (total area=4.5 mm²) and 8 on the right (total area=3 mm²) segment of the cribriform plate. The conductivities of tissues are 0.126, 0.276, 1.65, 0.01, 0.465, 2.5×10−14 S/m for white matter, gray matter, CSF, bone, skin, and air respectively. In addition, since olfactory nerves pass through the cribriform foramina, the corresponding voxels are assigned the conductivity of nerves (0.39 S/m). The tissue segmentation is shown in FIG. 3. The air-filled ethmoid labyrinth, sphenoid sinus, and frontal sinus are also shown. The colors are indicative of different materials: dark blue: white matter, blue: gray matter, cyan: CSF, green: bone, yellow: skin, orange: air, red: nerve respectively.

The transnasal electrodes may be used in any combination with external electrodes placed on the scalp. In one embodiment, 32 electrodes were placed on the scalp according to the standard “10-20” system. In addition, 5 electrodes were placed inside each half of the sphenoid sinus with a spacing of at least 5 mm, adjusting as required to the sphenoid sinus anatomy, for placement in contact with the sinus wall. 4 electrodes were placed beneath the cribriform plate bilaterally in each hemisphere (8 total), separated by 5 mm along the anterior-posterior direction. The electrodes on both sides of the olfactory cleft are separated by the perpendicular plate of the ethmoid bone, with a distance of 1 cm.

The forward simulation is completed with the ROAST platform with adaptations that allow customized placement of transnasal/transoral electrodes and more realistic conductivity of skull-base bones. The automatic pipeline includes tissue segmentation from MRI scans, conductivity value assignment, electrode placement, 3D meshing, and solving with the finite element method (FEM).

FEM simulations were used to solve the Laplace equation, where the electric field along each of the x, y, and z directions at brain voxels (E∈R^{p×1}) and currents applied by electrodes (s∈R^{n×1}) are related by:

$$E = As \qquad ( 1 )$$

where:
n is the number of electrodes; and
p is the thrice the number of brain voxels.

Because the total current in must equal the total current out, the entries of s must sum up to 0. Here, A∈R^{p×n} is the forward matrix, derived from concatenating the results of the n forward simulations column-wise, and there are three such Aₓ, A_{y}, A_{z} as the forward matrices for the resulting electric field along the x, y, z directions. In simulations, 18 transnasal electrodes are used (10 in olfactory cleft and 8 sphenoid sinus, split equally in the two hemispheres) and 32 scalp electrodes are used, for a total of n=50 transnasal and scalp electrodes. Because the desired targets are within the brain, the forward matrix only contains voxels in the brain (segmented as either gray matter or white matter), which leads to a total number of voxels of p=13,246,011.

To optimize the maximum intensity at the target, the assumption is made that the ROI is a single voxel in the target region. To target the ROI voxel with a large electric field along the desired direction (regardless of fields in other regions), the following optimization framework is used:

$$Max Intensity Framework : {u}^{T} {A}_{f} s \\ s . t . {1}^{T} s = 0 , {‖ s ‖}_{1} \leq 2 {I}_{safe} \qquad ( 2 )$$

The matrix A_{f} is the concatenation of the rows of Aₓ, A_{y}, A_{z} corresponding to the ROI voxel only. When multiplied by s, it yields the electric field in the x, y, z direction at the ROI. The unit vector u is the desired direction of the stimulation electric field, and taking the dot product yields the electric field along u. For subcortical regions (e.g., the thalamus, amygdala), u is chosen randomly. For cortical regions (e.g., the OFC), it is assumed that the optimal direction of stimulation is along the cortical column, so u is computed as a unit vector orthogonal to the cortical surface. The constraints make sure that Kirchhoff's current law is satisfied (i.e., the currents at the active and return electrodes sum up to 0) and the total injected current is bounded by a safety limit.

The method may also be optimized for focality of stimulation. To create focal fields at the target, the fields at non-target regions should be kept small. The following convex optimization framework is used to encourage focality:

$$Max Focality Framework : arg {argmin}_{s} {‖ {A}_{c} s ‖}_{2}^{2} \\ s . t . {1}^{T} s = 0 , {‖ s ‖}_{1} \leq 2 {I}_{safe} , {u}^{T} {A}_{f} s = E \qquad ( 3 )$$

In the objective, A_{c}∈R^{p×n} is the forward matrix corresponding to non-ROI rows, and Aₑs yields the electric fields at voxels outside the ROI. Minimizing the L2 norm minimizes the (non-directional) magnitude of electric fields at non-ROI regions. The additional equality constraint specifies that the directional intensity at ROI voxels reaches a desired value E (e.g., the neural threshold for stimulation). While the maximum intensity is optimized in the objective, here it is manually input into the constraint. E determines the operating point on the intensity-focality trade-off: small E results in focal fields that are lower intensity, and large E induces high-intensity but diffused fields. Note that, if E is too large, the optimization problem becomes infeasible.

Because the objective is quadratic and the number of voxels is large, the optimization problem is computationally expensive. For computational efficiency, singular value decomposition of the matrix Ac for optimization was performed (i.e., A_{c}=UΣV). Therefore, the optimization objective can be rewritten as:

$${‖ EV ‖}_{2}^{2} \qquad ( 4 )$$

This is because ∥A_{c}s∥₂²=∥UΣVs∥₂²=∥ΣVs∥₂². The second equality holds because U is unitary. Note that previously, A_{c} is a p×n matrix, where p»n. Here ΣV has a dimension of n×n, which makes the computation more efficient.

The quantitative metrics for determining intensity and focality will now be discussed. The simulation of the disclosed method has 50 electrodes in its search space, while the scalp-only configuration has 32 scalp-electrodes. Stimulation performance is assessed based on metrics of “Intensity Gain” and “Focality”. The Intensity Gain is the ratio of the electric-field intensity attained by the method and scalp-only at ROI along the desired direction:

$$\frac{{E}_{scalp + transnasal}}{{E}_{Scalp only}}$$

Focality is quantified using three metrics. define PV ER is defined as the percentage of voxels (inside the brain) with electric-field magnitude exceeding that of the ROI voxel. This metric is analogous to the commonly used full-width-half-max (FWHM), but is more suited to this method as the overall field maximum might not be attained in the ROI (e.g., when the ROI is not adjacent to any electrode). Second I(k) is computed, which is the integral of the electric field magnitude included within the k-nearest brain voxels of the ROI, and

$${I}_{frac} ( k ) = \frac{I ( k )}{I ( p )} ,$$

the ratio of the integral I(k) to the integral of the field in the entire brain (note that field outside brain voxels is not included in this integral). Finally, the Kvalue, (K₅₀) from the second metric, where K₅₀ is the threshold value of k for which I_{frac}(k)=0.5, (i.e., the value of k at which 50% of the total field is reached. For consistency, the total injected current (i.e., I_{safe}) was fixed to 1 mA for all simulations.

The cribriform plate of the ethmoid bone has a lot of variation across individuals, including the size and the number of formina. Monte Carlo simulations were performed to understand how the variations in the cribrifrom plate anatomy can affect the results. The NY Head tissue mask was modified for the cribriform plate foramina, where the area of each hole on each side of the cribriform plate is drawn uniformly in the interval [3,9] mm², and the number of holes on each side is drawn uniformly between 7 and 15. With this sampling, a total of 10 independent cribriform plate anatomy samples were generated. Due to their location and proximity to olfactory cleft electrodes, electric fields in the brain region close to the cribriform plate, namely, the medial anterior OFC, is expected to be most sensitive to changes in cribriform plate anatomy. Therefore, in the Monte Carlo simulations, an active electrode at the olfactory cleft electrode and the return at FCz, targeting medial anterior OFC was used.

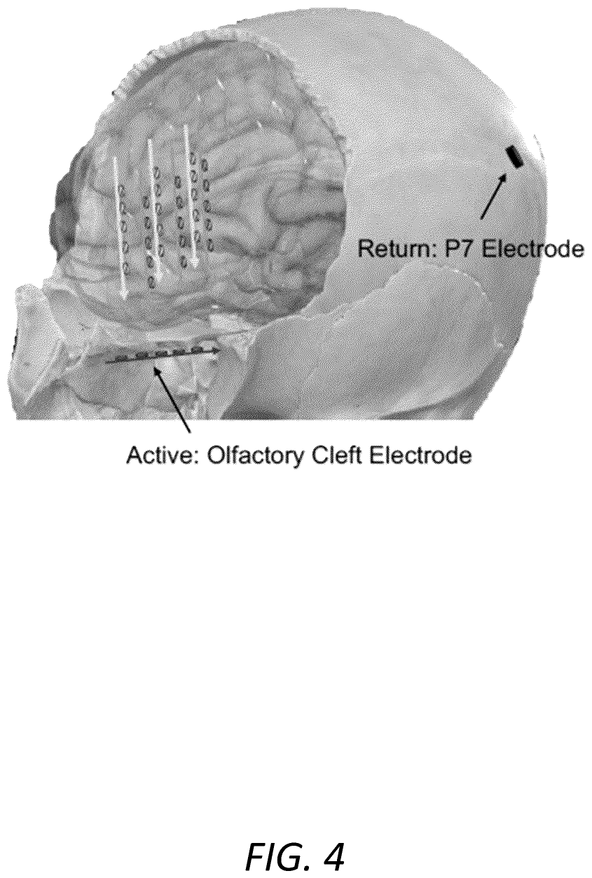

To test the efficiency of the method, cadaver studies were performed with specimens obtained from the Anatomy Gifts Registry. A neurosurgeon inserted three stereoencephalography (sEEG) probes roughly 1 cm apart, each with multiple electrodes, in each hemisphere, reaching just above the cribriform plate. The transnasal electrodes were inserted with endoscopic guidance, bilaterally in the olfactory cleft and above the superior turbinate, directly below the cribriform plate, as shown in FIG. 4. Six recording probes were inserted bilaterally (i.e., 3 in each hemisphere) to reach the medial OFC from above. The stimulation sEEG electrodes were inserted bilaterally below the cribriform plate through the nose using endoscopic guidance. The return electrode P7 was placed according to the 10-20 system. The placement of electrodes was verified by CT scanning.

Gold-cup electrodes were placed on the scalp at locations FP2, P7 and Pz according to the EEG 10-20 system. Stimulation pulses were applied to selected electrode pairs (nasal electrode and/or scalp electrode), while the resulting voltage at the brain electrodes was recorded. Biphasic, 10 ms/phase pulses of 10 mA amplitude were injected using a commercial neurostimulator. Pulses were repeated at 10 Hz, and the resulting fields averaged over 300-600 pulses. The electric field inside the brain was estimated as the gradient of the measured potentials along the sEEG probes.

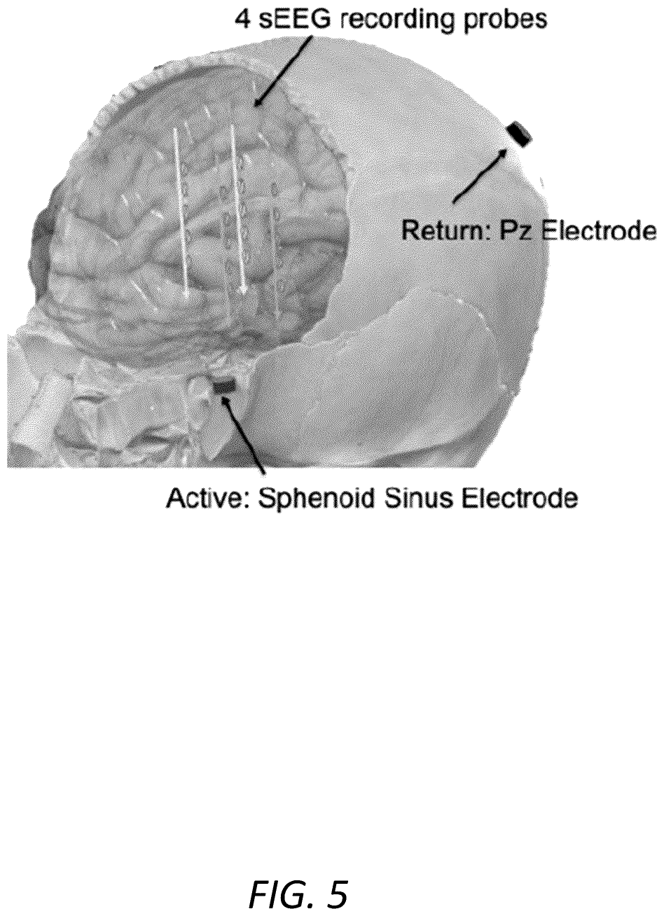

Currents were also injected using an electrode in the sphenoid sinus. As shown in FIG. 5, an electrode was placed inside the left sphenoid sinus, in contact with the superior wall of the sphenoid sinus. The return electrode was placed at Pz on the scalp. Four sEEG probes were inserted for recording.

The results of the simulation were as follows. Targets of stimulation are sampled from the anterior, posterior, medial, and lateral OFC at random locations. For each region, 10 randomly sampled target ROIs (one voxel each) are utilized in the estimates. The ROI of each sample is a single voxel within the region and the optimal direction for each sample is assumed to be perpendicular to the cortical surface. To implement this, we compute the unit vector perpendicular to the closest voxelized gray matter surface.

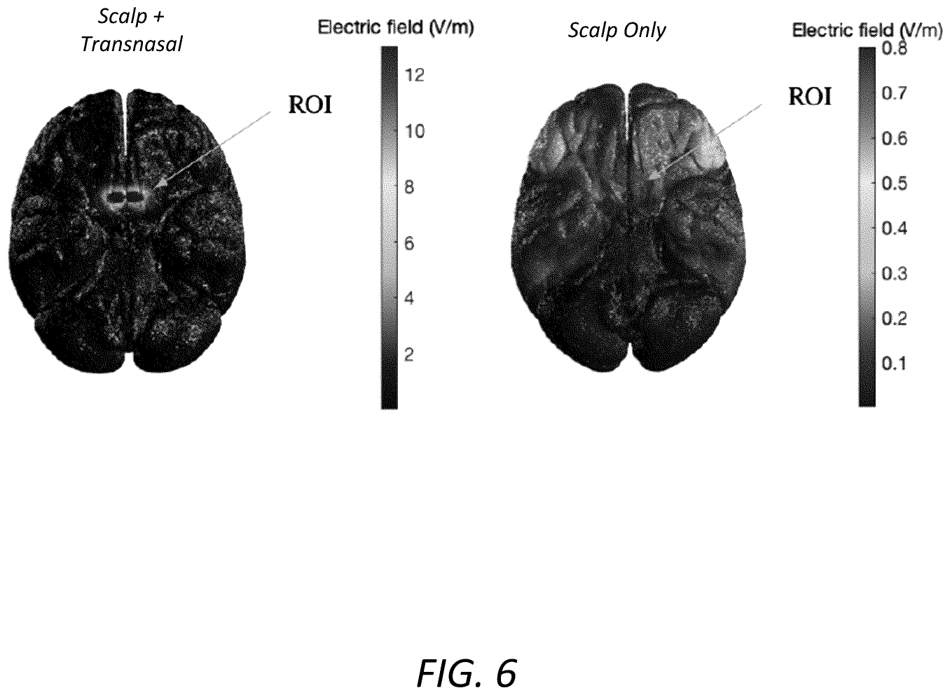

To quantify the intensity gain, the previously-discussed Max Intensity Framework was utilized for optimization. Because electrodes are placed in close proximity to the medial-posterior OFC, it can be targeted with the largest field intensity. In contrast, with scalp-only configurations, medial-posterior OFC has very small field intensities. The maximum intensity gain is 102.3 at a medial posterior ROI. To target that ROI voxel, a combination of electrodes from sinuses on both sides are used, and the resulting electric field is shown in FIG. 6.

In scalp-only configurations, optimization with the Max-Intensity Framework utilizes the forehead electrodes F7 and F8 to target the ROI. Despite no explicit constraints of focality in the optimization framework, more focal field was created at the OFC, and scalp-only configuration activates shallow regions outside the target mostly near the electrodes.

For the focality-constrained optimization for the 10 randomly selected OFC targets, the previously-discussed Max Focality Framework was utilized. The ROI is a single voxel, and the cancel region (i.e., voxels with minimized field) are the voxels excluding the ROI and its k-nearest voxels.

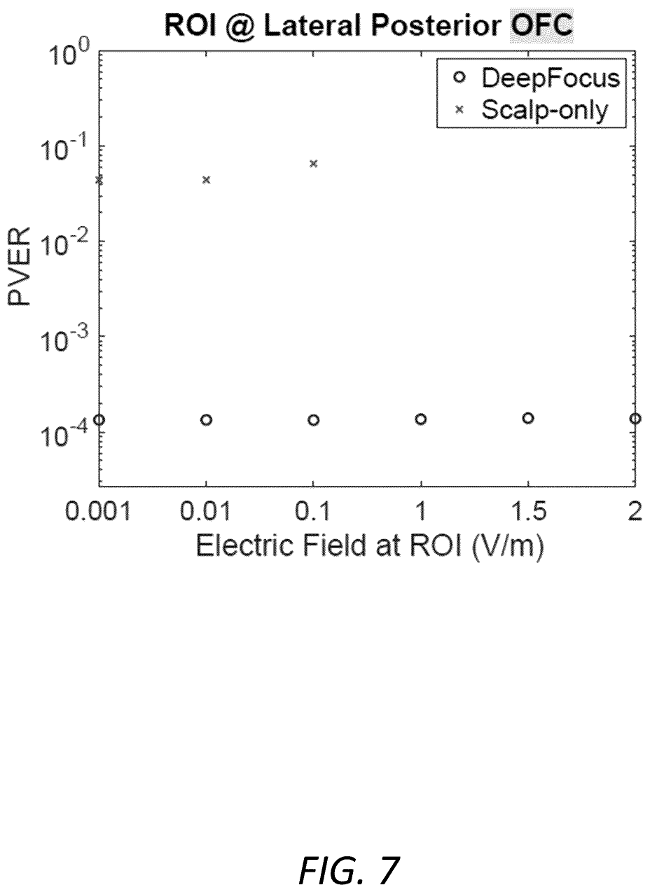

For the reported results, k=1000 was used, which is equivalent to a volume of 125 mm³. FIG. 7 shows the number of voxels with field magnitude greater than that of ROI (i.e., PV ER) for both scalp-only and scalp+transnasal configurations as the desired intensity increases along the x-axis. The electric fields created by scalp-only electrodes are more diffused than those by transnasal configurations. Additionally, scalp-only optimization problems are unable to reach the desired intensity at ROI with a much lower target intensity value, given the total current constraint.

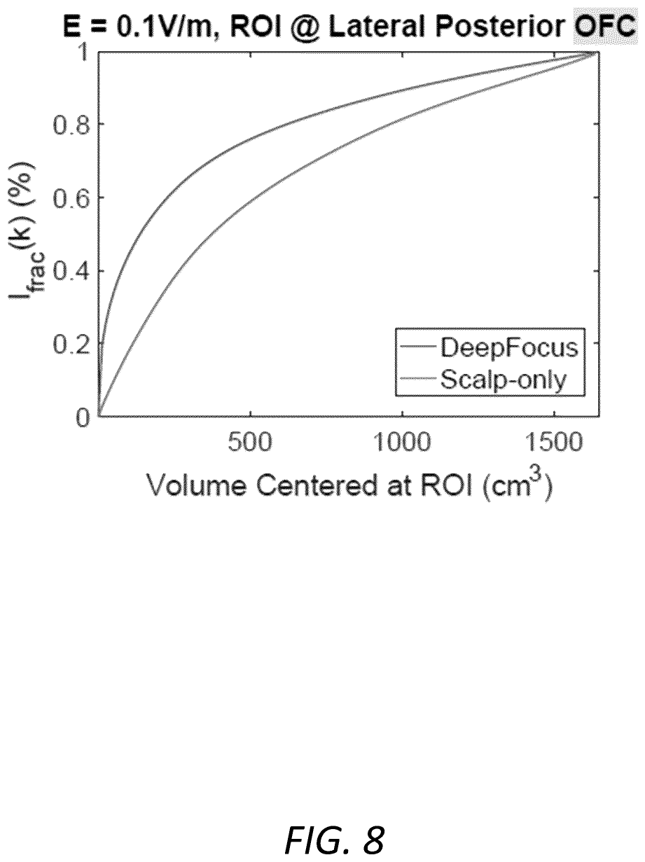

FIG. 8 shows the percentage of fields included in an increasing volume around the ROI at OFC (i.e., I_{frac}(k)). The desired intensity at the target is set to a nominal 0.1 v/m corresponding to the largest intensity that is feasible using scalp-only configurations. Depending on the feasible current (e.g., 15 mA or more), the resulting electric field can be easily estimated by scaling the nominal field in a linear fashion.

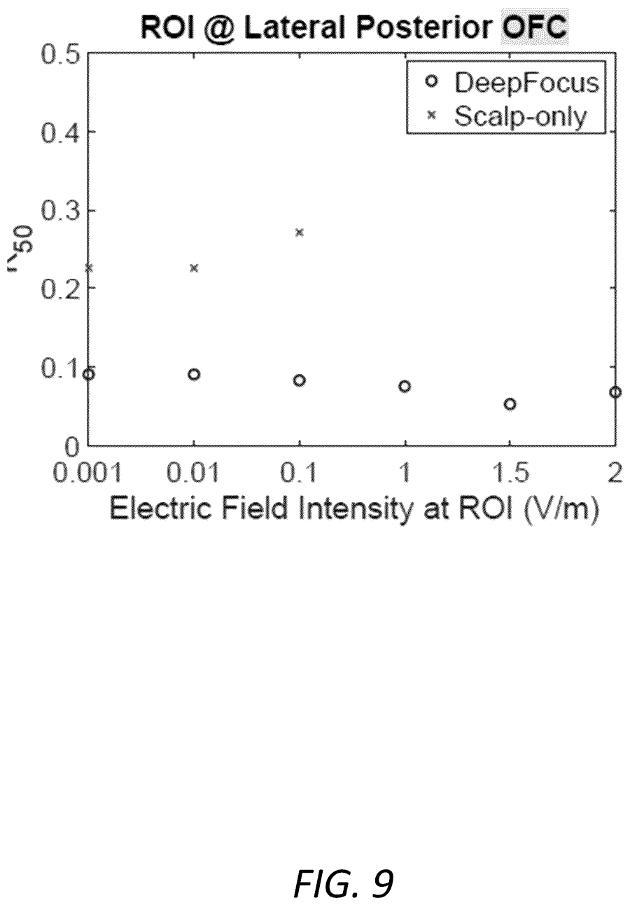

It can be observed that the percentage of voxels with magnitude higher than that of the ROI voxel is much larger with scalp-only configurations, indicating more diffused fields, as shown in FIG. 7, wherein the curve corresponding to the scalp+transnasal configurations has a much larger slope at smaller intensities and plateaus faster than scalp-only configurations, indicating that scalp+transnasal fields concentrate much more around the ROI, and thus have better focality. This is also illustrated in FIG. 9, where the K₅₀ value is lower for scalp+transnasal configurations than for scalp-only configurations, which indicates that 50% of the total field magnitude concentrates in a more localized brain volume.

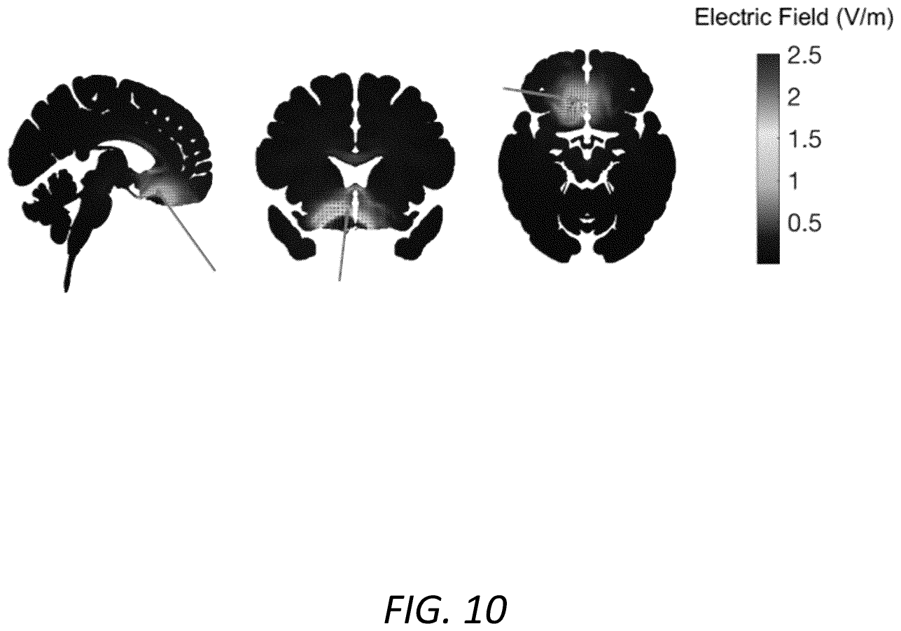

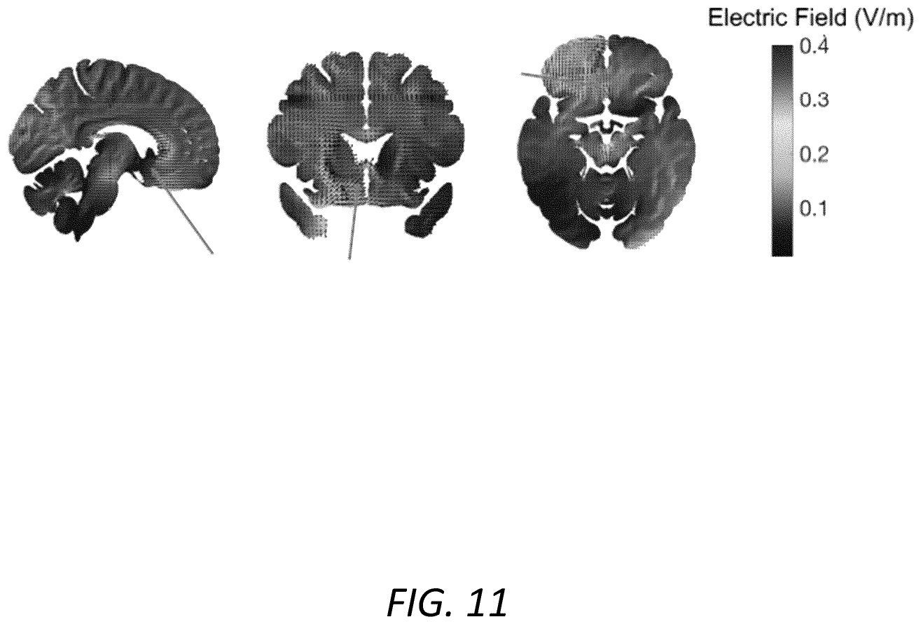

Brodmann Area 25 (BA25) is an important target for treatment-resistant depression. As with the OFC, 10 ROIs were randomly selected within BA25 bilaterally. With the previously-discussed Max-Intensity Framework, scalp+transnasal configurations obtain a maximum of 15.3× intensity gain over scalp-only configurations. FIG. 10 and FIG. 11 illustrate a comparison of scalp+transnasal patterns and scalp-only patterns targeting a voxel in BA25. In FIG. 10, an electrode inside the left sphenoid sinus is utilized to target the ROI. In scalp-only configurations, due to the medial location of BA25, an active electrode at Fp1 is used, with return at P4, to create fields traversing through BA25. Relative to the scalp+transnasal configuration, the optimized scalp-only configuration results in much more diffused fields and undesired activation of shallower brain regions near the scalp electrodes.

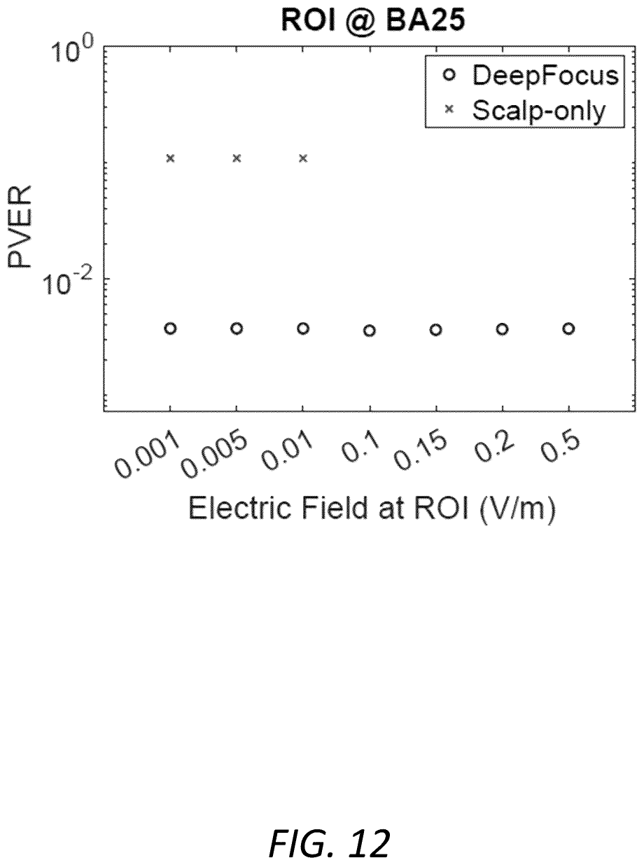

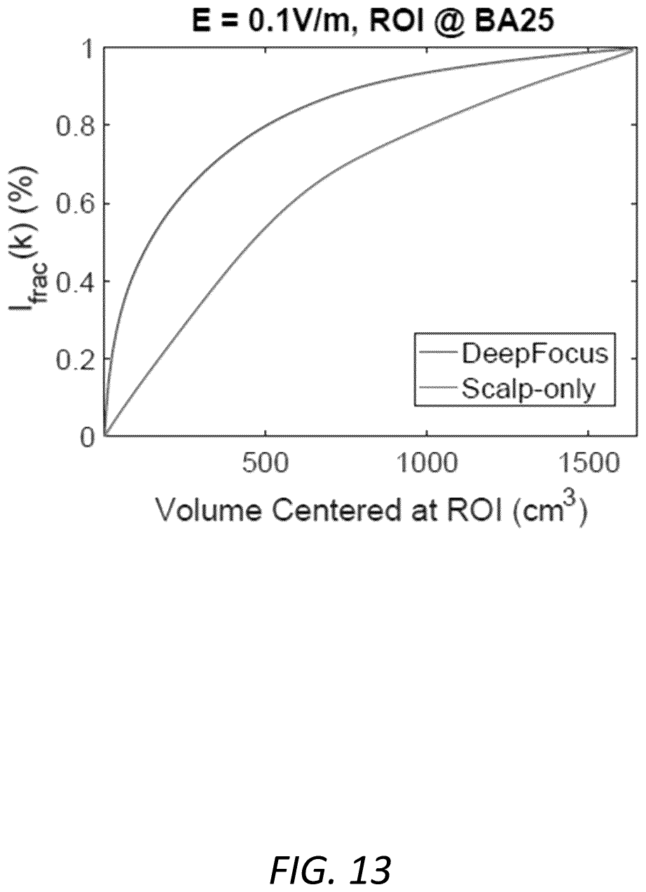

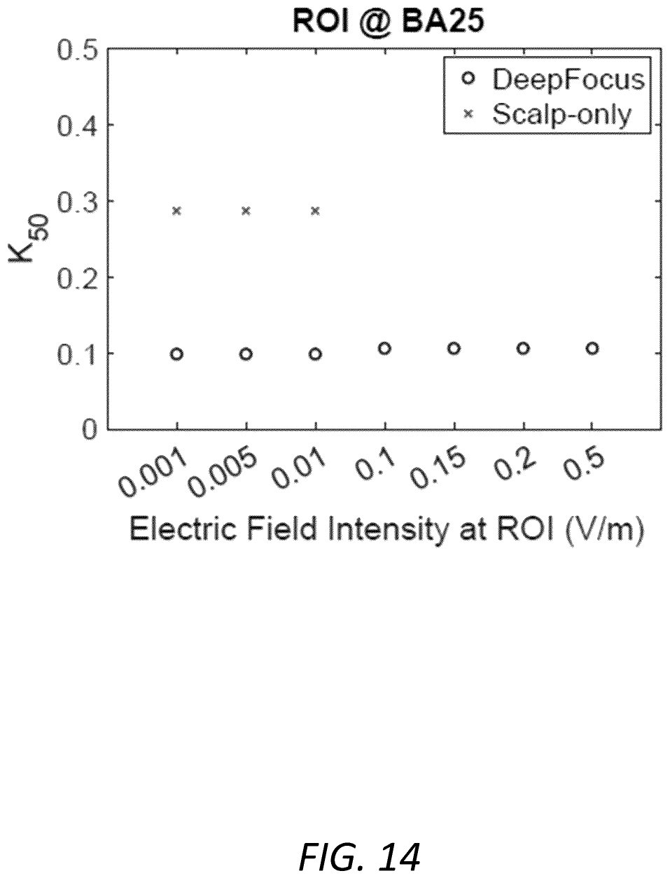

With the constraint of focality, the performance of scalp+transnasal and scalp-only configurations for BA25 targets are compared. As shown in FIGS. 12-14, the scalp+transnasal configurations induce more focal fields at BA25 targets. However, because BA25 is not as close to the transnasal electrodes as OFC, the gain in focality is smaller.

With the Max-Focality Framework, the optimal current injection patterns for scalp+transnasal and scalp-only configurations were determined. The intensity gain for targeting the ROIs in reward circuit nodes is shown in Table 1, including OFC, BA25, amygdala, nucleus accumbens, anterior ventral thalamus, and anterior hippocampus. 40 ROIs were sampled randomly in the OFC, and 10 ROIs were sampled randomly at other regions shown below. Among the ROIs in OFC, the optimization for both Max-Intensity and Max-Focality tends to select electrodes in the sphenoid sinus for posterior OFC targets, and olfactory cleft electrodes for targets in the anterior OFC (due to their proximity to the OFC).

TABLE 1
Intensity Gain in Reward Circuit Nodes

| | | | | | Hippo- | |
|---|---|---|---|---|---|---|
| Target ROI | OFC | BA25 | [symbol] | Nucleus [symbol] | campus | Thalamus |
| Maximum Gain | 1 [symbol] 2.3 | [symbol] | 7.9 | [symbol] .2 | 12.5 | 2.9 |
| Median Gain | 6.2 [symbol] | 8 | 4.4 | [symbol] .0 | 6.31 | 2.2 |

[symbol]

indicates data missing or illegible when filed

For each node, a benefit in intensity and focality gain was observed using scalp+transnasal configurations, even though the precise benefit is different for different regions based on their proximity to the electrodes.

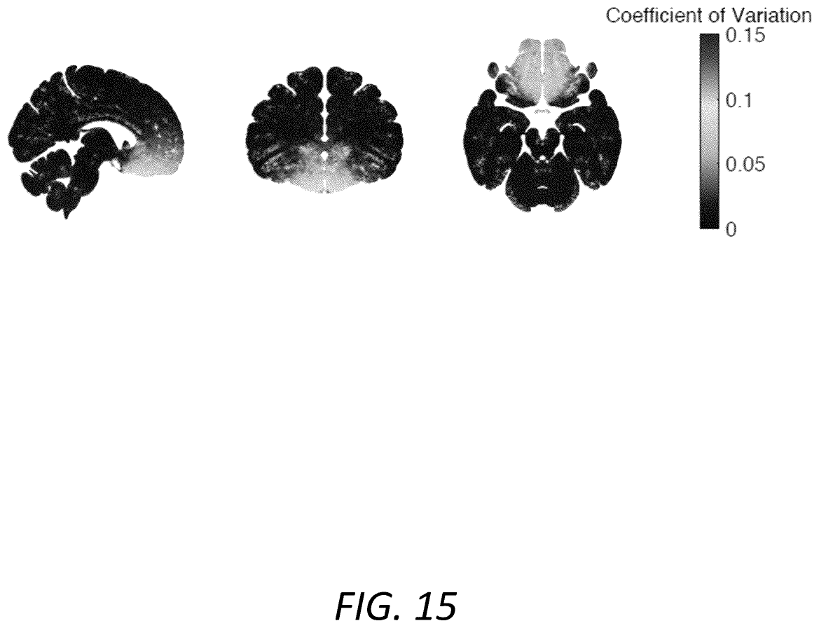

As previously described the cribriform plate anatomy was modified, and across the 10 different head models, the coefficient of variation (i.e., standard deviation mean) for every voxel inside the brain was computed using a Monte Carlo simulation analysis, as shown in FIG. 15. Slice views of the coefficient of variation in brain voxels are shown. Note that the variation of electric field magnitude is highly concentrated around the OFC region. The results suggest that the variation is predominantly limited to the OFC region, where the differences in the foramina sizes and locations induce the most variability. Overall, the volume of brain voxels that have more than 10% coefficient of variation is relatively small, 960 mm³, which is less than 1 cm³. If very precise stimulation in medial OFC is desired, this variation could require tailoring to the specific cribriform plate anatomy. In other regions, the effects might be too small to be relevant.

In the simulation results previously presented, scalp+transnasal configurations create higher intensity and more focal fields than scalp-only configurations. Using cadaver studies, a ROI at the medial OFC was simulated, and the fields in cadaver tissues were recorded.

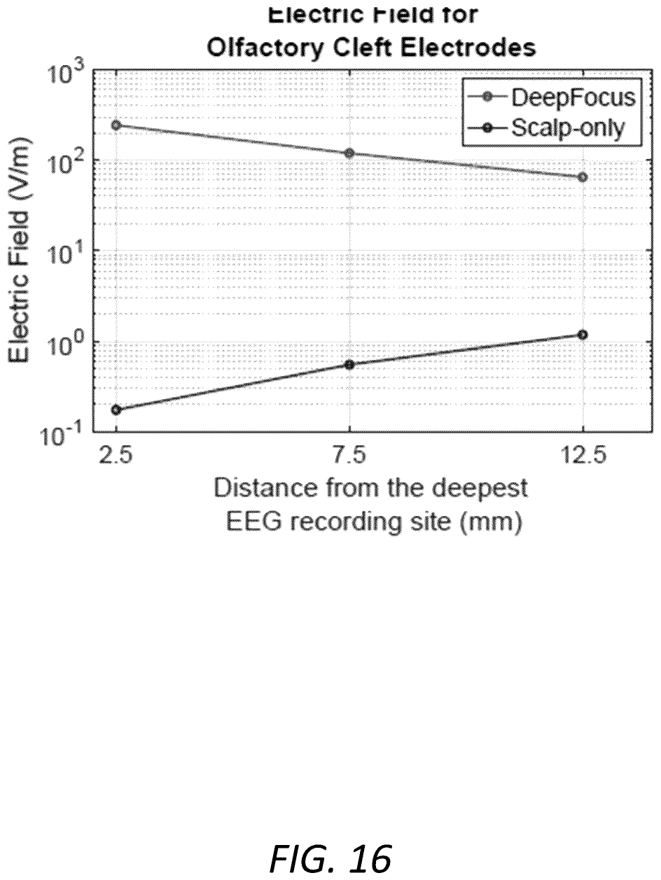

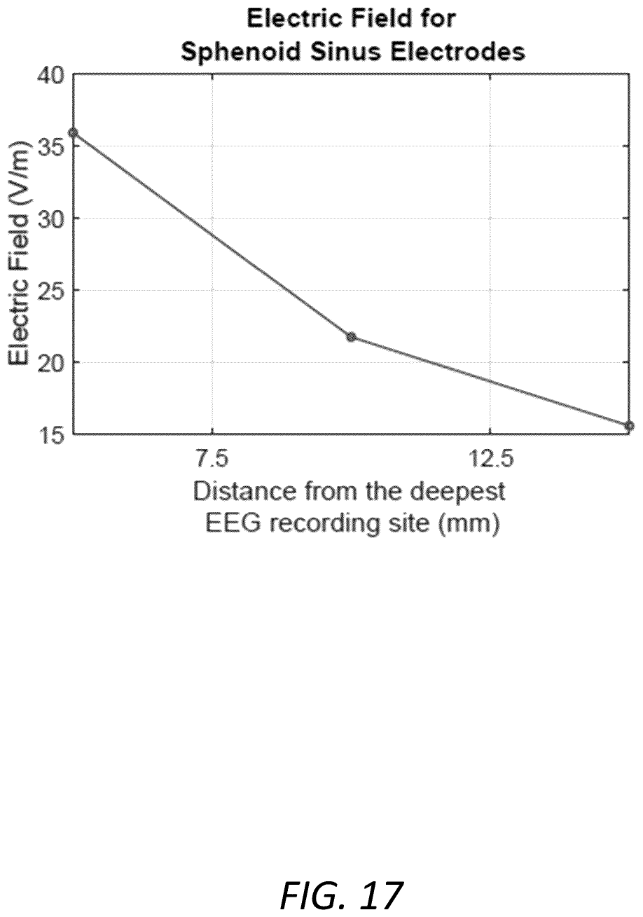

The placement of electrodes was selected based on the optimization results with the Max-Intensity Framework for a medial OFC target. For scalp+transnasal configurations, the optimization results suggested the utilization of an electrode inside the olfactory cleft and a return electrode at P7. FIG. 16 shows the estimated electric field in the direction of a single sEEG probe for both scalp+transnasal patterns and scalp-only patterns. The scalp+transnasal pattern (olfactory cleft and P7 electrodes) created much larger field intensity near OFC than the scalp-only pattern. Moreover, the field intensity attenuated rapidly when moving further away from medial OFC, inducing very small field intensities at more shallow regions, consistent with the simulations. FIG. 17 shows the recorded electric field as a function of distance from the deepest EEG recording site.

Simulations were performed for the current injection patterns utilized in the cadaver studies. For the sphenoid sinus stimulation, the simulated field at the locations of the sEEG probes is consistent with the recorded field. For the olfactory stimulation, simulations predict a smaller field than the recorded field, although within the same order of magnitude. This may be due to the cadaver specimen's cribriform plate being thinner than that of the New York head model, as quantified by examination of the cadaver CT scan and the New York head model segmentation.

Method for supplementing scalp electrode configurations with the placement of transnasal/transoral electrodes for targeting specific ROIs in the brain has been disclosed. The method can be used as a minimally-invasive technique to stimulate, for example, reward circuit nodes, in deep brain regions. The efficacy of the method has been shown through simulations comparing the efficiency and focality of scalp+transnasal/transoral patterns with traditional scalp-only TES patterns. For the same total injected current, scalp+transnasal/transoral patterns create larger and more focused fields at many different ROIs in the deep brain. The method is especially useful as a supplement to methods for optimizing electrode placement to target specific ROIs in the brain, such as the method disclosed in U.S. patent application Ser. No. 18/021,257.

1. A method comprising:
placing one or more electrodes on an exterior surface of the skull;
placing one or more electrodes into a cavity in the interior of the skull transnasally or transorally; and
delivering stimulation to a target brain structure using the exterior and interior electrodes.

2. The method of claim 1 wherein at least some of the transnasally/transorally-placed electrodes are placed under the cribriform plate.

3. The method of claim 1 wherein electrodes placed under the cribriform plate direct energy along pathways defined by foramina in the cribriform plate.

4. The method of claim 1 wherein at least some of the transnasally/transorally-placed electrodes direct energy along pathways defined by foramina in the skull base.

5. The method of claim 1 wherein at least some of the transnasally/transorally-placed electrodes are placed in the olfactory cleft.

6. The method of claim 1 wherein at least some of the transnasally/transorally-placed electrodes are placed in one or more of the frontal, ethmoidal and sphenoid sinuses.

7. The method of claim 1 wherein the transnasally/transorally-placed electrodes are disposed on a balloon that can be inflated after insertion into the cavity.

8. The method of claim 1 wherein the transnasally/transorally-placed electrodes are configured as a 2-dimensional or 3-dimensional array.

9. The method of claim 1 wherein the transnasally/transorally-placed electrodes are battery powered or powered wirelessly.

10. The method of claim 1 wherein the stimulation is an electric field

11. The method of claim 10 wherein electric field is steerable after insertion of the transnasally/transorally-placed electrodes.

12. The method of claim 11 wherein the electric field is steered using a beamforming process.

13. The method of claim 1 wherein the stimulation is electronic, magnetic, light, thermal or ultrasound.

14. The method of claim 1 wherein placement and activation of the externally-placed electrodes and the transnasally/transorally-placed electrodes may be optimized using machine learning models.

15. The method of claim 1 wherein the transnasally/transorally-placed electrodes are also used for sensing a response to the stimulation.

16. The method of claim 1 wherein the transnasally/transorally-placed probes are used for delivering light, ultrasound, to the brain or cranial nerves.

17. The method of claim 1 wherein the transnasally/transorally-placed probes are used for delivering or wireless power to a brain implant.

18. The method of claim 11 wherein the stimulation may be directed to specific voxels within a brain.

19. The method of claim 16 wherein intensity of the stimulation may be optimized for a specific voxel within a brain.

20. The method of claim 16 wherein focality of the stimulation may be optimized for a specific voxel within a brain.
